(19) United States
(12) Patent Application Publication
(10) Pub. No.: US 2003/0109901 A1
(43) Pub. Date: Jun. 12, 2003

(54) Photonic pacemaker-cardiac monitor

(76) Inventor: Wilson Greatbatch, Akron, NY (US)

Correspondence Address: GREENWALD & BASCH, LLP, 349 WEST COMMERCIAL STREET, SUITE 2490, EAST ROCHESTER, NY, 14445, US

(21) Appl. No.: 10/014,890

(22) Filed: Dec. 11, 2001

(51) Int. Cl. A61N 1/362

(52) U.S. Cl. 607/9

(57) ABSTRACT

A photonic pacemaker-cardiac monitor apparatus for use during an MRI procedure includes a photonic pacemaker adapted to pace an MRI patient's heart via a photonic catheter, an electrocardiagraphic monitor adapted to sense cardiac electrical activity via the photonic catheter, an oxygen monitor adapted to sense cardiac blood oxygen content via the photonic catheter, and a warning system for warning of a danger condition wherein one or more of the following occurs: 1) the patient fails to receive proper pacemaker stimulation; 2) the patient fails to exhibit proper cardiac electrical activity; or 3) the patient fails to exhibit proper cardiac mechanical activity. The warning system may include a display for providing a visual indication of outputs from the pacemaker, the electrocardiagraphic monitor and the oxygen monitor. The apparatus is fully compatible with MRI diagnostic procedures. It preferably includes a wearable housing having a control panel with three flashing lights providing the display. The first light flashes when a pulse is delivered by the photonic pacemaker. A second flashing light occurs about a tenth of a second after the first flashing light when the first cardio-monitor senses R wave activity in the heart. The third light operates when the second cardio-monitor senses oxygenated blood and thus mechanical activity of the heart. Thus, there will be a sequence of three flashing lights indicating that a pacing signal is being applied to the heart and the heart is responding with an electrocardiographic R wave and with a pulsatile blood flow. This will enable an attending physician to, at a glance, see many of the vital functions of the heart so as to better monitor the patient's response to the MRI procedure.

### BACKGROUND OF THE INVENTION

1. Field of the Invention

The present invention relates to pacemakers. More particularly, the invention concerns MRI compatible pacemakers with cardiac monitoring capability for use during MRI diagnostic procedures.

2. Description of Prior Art

By way of background, pacemakers for delivering stimulating electrical energy to the heart, “R” wave amplifiers for sensing the heart's electrical activity, and oxygen sensors for sensing the heart's blood oxygen content (and hence its mechanical functionality), are all known in the art, both separately and in combination. As far as known, however, what has not been available is an apparatus that combines the foregoing functionality in a system which is adapted for use in an MRI diagnostic environment, and which allows a medical practitioner to directly monitor a pacemaker patient's cardiac response during MRI treatment. Indeed, the use of any form of pacemaker device is generally contraindicated for pacemaker patients, as described by way of background in copending application Serial Nos. 09/864,944 and 09,865,049, both filed on May 24, 2001, and in copending application Serial Nos. 09/885,867 and 09/885,868, both filed on Jun. 20, 2001. In these copending patent applications, each of which names applicant as a co-inventor, and whose contents are fully incorporated herein by this reference, MRI compatible/safe pacemakers are disclosed for both implantable and wearable use. The disclosed pacemakers feature photonic catheters carrying optical signals in lieu of metallic leads carrying electrical signals in order to avoid the dangers associated with MRI-generated electromagnetic fields. In addition, only non-ferromagnetic materials and a minimal number of metal components of any kind are used.

Despite the advances in pacemaker MRI compatibility and safety offered by the devices of the above-referenced copending applications, there remains an unsatisfied need for an MRI compatible pacemaker that includes electrical and oxygen sensing capability, and which is particularly adapted for MRI use so as to enable a medical practitioner to directly monitor a patient's cardiac activity during MRI scanning. What is required is an improved photonic pacemaker cardiac monitor that is capable of withstanding the strong magnetic and electromagnetic fields produced by MRI equipment without operational disruption and without producing physiological injury due to magnetically induced mechanical movement and electromagnetically induced electrical current. Additionally, the apparatus should provide reliable real-time information concerning cardiac activity to advise a medical practitioner during MRI scanning of any abnormalities in cardiac function, thereby allowing the practitioner to take immediate responsive action.

### SUMMARY OF THE INVENTION

The foregoing problems are solved and an advance in the art is provided by a photonic pacemaker-cardiac monitor apparatus that includes a photonic pacemaker adapted to pace a heart via a photonic catheter, an electrocardiagraphic monitor adapted to sense cardiac electrical activity via the photonic catheter, an oxygen monitor adapted to sense cardiac blood oxygen content via the photonic catheter, and a warning system for warning of a condition wherein one or more of the following occurs: 1) the patient fails to receive proper pacemaker stimulation; 2) the patient fails to exhibit proper cardiac electrical activity; or 3) the patient fails to exhibit proper cardiac mechanical activity. The warning system can be implemented as a display for providing a visual indication of outputs from the pacemaker, the electrocardiographic monitor and the oxygen monitor, and/or an audio warning can be generated. Optionally, a core body temperature sensor and an associated visual display indicator may also be added to the photonic pacemaker-cardiac monitor apparatus.

The apparatus can be embodied using three enclosures that may comprise an exemplary implementation of the apparatus, namely, a wearable external control housing located at a proximal end of the photonic catheter, a first distal housing located at the distal end of the photonic catheter, and a second distal housing located next to, but spaced from, the first distal housing.

The photonic pacemaker preferably comprises an electronic pulse generator and an electro-optical converter situated in the control housing, a first optical conductor running through the photonic catheter, and an opto-electrical converter situated in the first distal housing. The ring and tip electrodes may be respectively provided by the first and second distal housings themselves.

The electrocardiagraphic monitor preferably comprises an EKG amplifier and an electro-optical converter situated in the first distal housing, a second optical conductor running through the photonic catheter, and an opto-electrical converter and amplifier situated in the control housing.

The oxygen monitor preferably comprises an oxygen sensor situated in the first distal housing, a possible electro-optical converter located in the first distal housing (depending on the type of oxygen sensor used), a third optical conductor running through the photonic catheter, and an opto-electrical converter and amplifier situated in the control housing.

If a visual display is present, it can be implemented using three flashing lights mounted on a control panel of the control housing. The first flashing light indicates that an optical pulse has been delivered by the pacemaker. The second flashing light, which would closely follow the first flashing light, indicates that there is electrocardiographic activity resulting from the stimulation supplied by the pacemaker. The third flashing light indicates that there is not only electrical activity in the heart in response to the stimulating signal, but also mechanical activity. The sequential flashing of the three lights indicates that the heart is being stimulated successfully. By glancing at the visual display on the control housing, a medical practitioner will be provided with a quick view of this information, and in this way the patient can be closely monitored for MRI induced abnormal cardiac activity during an MRI procedure.

The photonic pacemaker-cardiac monitor apparatus thus provides a stand-alone cardiac stimulating and monitoring system. MRI compatibility is derived from the fact that there are no electrical metallic conductors going from the external control housing to the heart. The signals and power are carried via the photonic catheter and, wherever necessary, transformed back to electrical signals or vice versa.

### BRIEF DESCRIPTION OF THE DRAWINGS

The foregoing and other features and advantages of the invention will be apparent from the following more particular description of preferred embodiments of the invention, as illustrated in the accompanying Drawing in which:

FIG. 1 is a block diagrammatic view of a photonic pacemaker-cardiac monitor constructed in accordance with a preferred embodiment of the present invention;

FIG. 2 is a diagrammatic view of a first oxygen sensor for use in the apparatus of FIG. 1; and

FIG. 3 is a diagrammatic view of a second oxygen sensor for use in the apparatus of FIG. 1.

### DETAILED DESCRIPTION OF PREFERRED EMBODIMENTS

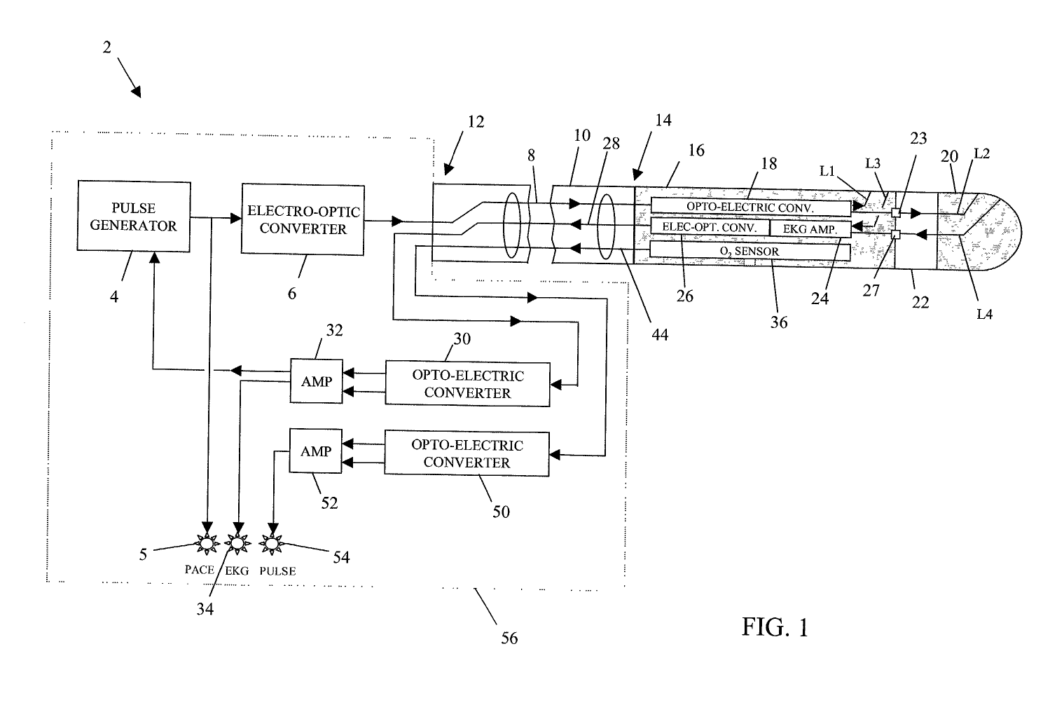

Turning now to FIG. 1, a photonic pacemaker cardiac monitor apparatus 2 is shown. The apparatus 2 comprises an electronic pulse generator 4 that produces electrical pulses at its output. The electrical pulses drive the input of an electro-optical converter 6, which may be implemented as a laser diode light generator, such as a gallium arsenide laser, or alternatively, as a light emitting diode. The electrical pulses from the pulse generator circuit 4 are also fed to an indicator light 5 (e.g., a light emitting diode or the like) that flashes in correspondence with the pulses. The electro-optical converter 6 generates optical pulses at its output in correspondence with the electrical pulses output by the pulse generator 4. The optical pulses are impressed onto an optical conductor 8 (e.g., a fiber optic element) situated in a photonic catheter 10 that extends from a proximal end 12 to distal end 14 thereof. The distal end 14 of the photonic catheter 10 attaches to a first distal hermetic housing 16. There, the optical conductor 8 terminates at an opto-electrical converter 18 that is hermetically sealed within the first distal housing 16. The opto-electrical converter 18, which is preferably implemented as a photodiode array to develop the necessary photovoltaic electrical potential, converts the optical pulses into electrical pulses of approximately 3-4 volts at 4 milliamperes, which is capable of stimulating the implanted heart to beat.

The tip and ring electrodes that deliver the electrical pulses output by the opto-electrical converter 18 to the heart may be constructed in accordance with the disclosures of the copending patent applications referenced above. In particular, the first distal housing 16 can be configured to act as the ring electrode. The tip electrode can be provided by a second distal housing 20 that is separated from the first distal housing 16 by a short section 22 (e.g., about 0.5-1.0 inches) of a biocompatible electrically insulating material such as silicone rubber, polyurethane, polyethylene, or the like. In order to function as electrodes, the housings 16 and 20 are made from a suitable implantable electrode material that is also non-ferromagnetic, such as platinum, titanium, alloys or platinum or titanium, or the like. These components are electrically connected to the opto-electrical converter 18, as shown in FIG. 1, via electrical leads L1 and L2. The electrical lead L1 connects to the wall of the first distal housing 16. The electrical lead L2 exits the first distal housing 16 via a hermetic seal terminal 23, passes through the section 22, and connects to the wall of the second distal housing 20. When implanted in a patient's heart, the second distal housing 20 will preferably be embedded in the endocardial wall of the heart and driven negatively with respect to the first distal housing 16, which will preferably sit in the right ventricle in contact with the blood stream.

The foregoing components that drive the heart may be collectively referred to as a photonic pacemaker. When stimulated by the photonic pacemaker, the heart should adequately perform a blood pumping cycle. However, there is no guarantee that this will occur, especially when the patient is undergoing an MRI diagnostic procedure. Thus, the apparatus 2 provides two alternative sensing systems that respectively monitor the heart's electrical and mechanical activity. The first sensing system is an electrocardiagraphic monitor. The second sensing system is an oxygen monitor.

The electrocardiagraphic monitor begins with the same tip and ring electrodes used to stimulate the heart. Shortly after being driven by the photonic pacemaker, the tip and ring electrodes (i.e., housings 20 and 16, respectively) will pick up a resulting electrocardiographic “R” wave pulse signal (if it is present) from the implanted heart. This signal is amplified by a micro-miniature EKG amplifier 24 that is hermetically sealed within the first distal housing 16 and electrically connected to the tip and ring electrodes via electrical leads L3 and L4. The electrical lead L3 connects to the wall of the first distal housing 16. The electrical lead L4 exits the first distal housing 16 via a hermetic seal terminal 27, passes through the section 22, and connects to the wall of the second distal housing 20. The amplified “R” wave pulse output from the EKG amplifier circuit 24 drives an electro-optical converter 26 that is also hermetically sealed in the first distal housing 16. The electro-optical converter 26 is preferably implemented as a light emitting diode or other low cost device. A pulsatile optical signal is output from the electro-optical converter 26 and impressed onto an optical conductor 28 (e.g., a fiber optic element) situated in the photonic catheter 10. The optical pulses are delivered to an opto-electrical converter 30 (e.g., a photodiode) located at the proximal end of the photonic catheter 10 that converts the optical pulses into electrical pulse signals that are amplified by an amplifier 32. The electrical pulse signals from the amplifier 32 are fed to an indicator light 34 (e.g., a light emitting diode or the like) that flashes in correspondence with the pulses. The electrical pulse signals may also be fed back to the pulse generator 4 as part of a feedback circuit to control the pulse generator 4, e.g., by temporarily inhibiting the next stimulating pulse or by decreasing the pulse width of the next stimulating pulse to a point below which it could not possibly stimulate the heart. If no “R” wave appears, there is no inhibiting input applied by the feedback circuit and the next pulse from the pulse generator will be of the normal pulse width (approximately 1 millisecond) needed to drive the heart.

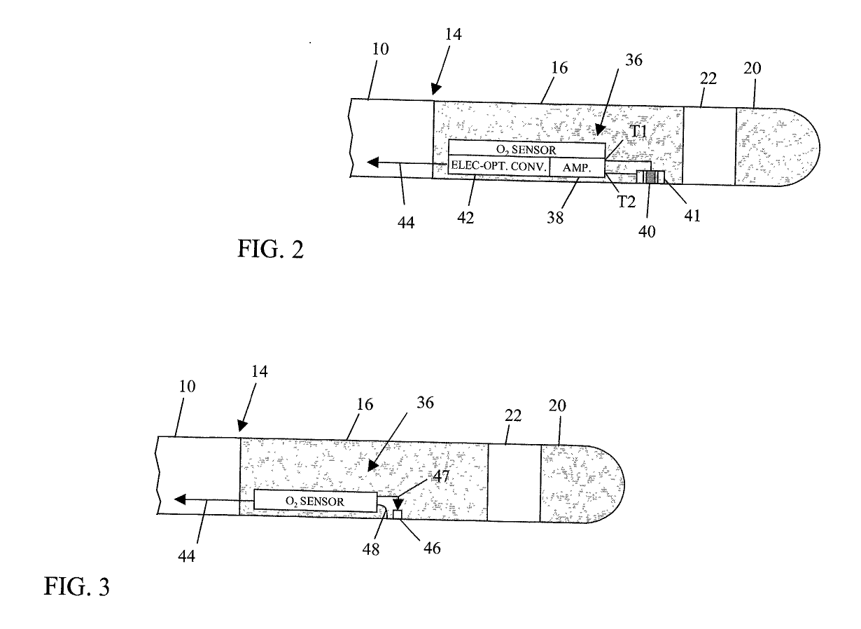

The oxygen monitor of the apparatus 2 begins with an oxygen sensor 36 that is partially hermetically sealed in the first distal housing 16. Two alternative constructions for the oxygen sensor 36 are illustrated in FIGS. 2 and 3. In FIG. 2, the oxygen sensor 36 is implemented as a conventional “Clark” electrode. In this configuration, a first terminal T1 of a micro-miniature amplifier 38 is electrically connected to a platinum electrode 40 whose cross-section is in contact with the patient's cardiac blood. A second terminal T2 of the amplifier 38 is connected to a silver electrode 41 of much larger cross-sectional size than the platinum electrode 40 and whose cross section is also in contact with the patient's cardiac blood. As shown in FIG. 2, the electrode 41 can be hollow and the electrode 40 can be concentrically nested therein. Other arrangements, such as a pair of spaced wire electrodes, could also be used. The amplifier 38 is powered by a suitable electrical power source, such as the opto-electrical converter 18. Alternatively, a dedicated opto-electrical converter (not shown) may be used that is associated with the oxygen sensor 36 and driven by an associated optical conductor (not shown) carried in the photonic catheter 10. A potential of negative 0.6 volts with respect to the silver electrode 41 is applied to the platinum electrode 40. The electrical current through a circuit comprising the electrodes 40 and 41 and the blood that bathes the electrodes is a linear function of the oxygen content of the blood. The amplifier 38 can be configured to deliver an amplified pulse output when the current through this circuit is at a level that is consistent with the presence of adequately oxygenated blood in the heart. The amplified pulse is provided to an electro-optical converter 42 (e.g., a light emitting diode), where it is converted to a pulsatile optical signal that is impressed onto an optical conductor 44 (e.g., fiber optic element) situated in the photonic catheter 10.

In FIG. 3, the oxygen sensor 36 is implemented as a conventional pulse oximeter. In this configuration, a light source 46 (e.g., the end of a fiber optic element, a light emitting diode, etc.) is situated on a wall of the first distal housing 16 so as to be capable of shining illuminating light pulses into the adjacent blood. The light source 46 is driven by a conductive element 47 that may conduct either light or electrical signals, depending on the nature of the light source 46. If the conductive element 47 delivers electrical signals, a suitable electrical power source, such as the opto-electrical converter 18 may be used. Alternatively, a dedicated opto-electrical converter (not shown) may be used that is associated with the oxygen sensor 36 and driven by an associated optical conductor (not shown) carried in the photonic catheter 10. If the conductive element 47 delivers light signals, the signals may be provided by an associated optical conductor (not shown) carried in the photonic catheter 10.

An optical receiver 48 (e.g., a fiber optic element), which may be formed as an extension of the optical conductor 44, is placed with its input located next to the light source 46 so as to receive light pulses that are transmitted through or reflected by the blood surrounding the light source 46 and the optical receiver 48. The oxygen content of the blood can be determined from this light. In particular, white light from the light source 46 can be shone through a liquid blood sample and received by the optical receiver 48. The light is then split between two different glass filters (not shown), each of which selects a portion of the light spectrum characteristic to low or high oxygen content in the blood. The oxygen content is a function of the ratio of the light intensity from each of the two filters. The output can be displayed as a go/no-go light flash, or by a digital readout on a display panel. Note that the filters could be located in the first distal housing 16, if desired.

Regardless of which oxygen sensor configuration is used, the oxygen sensing signal information is sent back in the form of a pulsatile optical signal to the photonic catheter's proximal end 12 (see FIG. 1). There, the optical pulses carried by the optical conductor 44 are delivered to an opto-electrical converter 50 (e.g., a photodiode) located at the proximal end of the photonic catheter 10 that converts the optical pulses into electrical pulse signals that are amplified by an amplifier 52. The electrical pulse signals from the amplifier 52 are fed to an indicator light 54 (e.g., a light emitting diode or the like) that flashes in correspondence with the pulses.

The components of the apparatus 2 that are located at the proximal end 12 of the photonic catheter 10 may be conveniently placed in a control housing 56 that may be worn by the patient or located at some other location where it can be directly observed by an attending physician during an MRI procedure. The photonic catheter 10 is implanted in the patient in conventional fashion. As the apparatus 2 operates under normal conditions during an MRI procedure, the indicator lights 5, 34 and 54 should flash in sequence. The indicator light 5 will illuminate first to indicate that an optical pulse has been applied to the photonic catheter 10. The indicator light 34 will illuminate second to indicate that the heart has responded with an electrocardiographic “R” wave. The indicator light 54 will illuminate third to indicate that there was also mechanical activity in the heart as demonstrated by the presence of a pulsatile oxygen sensing signal.

Collectively, the indicator lights 5, 34 and 54 provide a warning system for warning of a danger condition wherein one or more of the following occurs: 1) the patient fails to receive proper pacemaker stimulation; 2) the patient fails to exhibit proper cardiac electrical activity; or 3) the patient fails to exhibit proper cardiac mechanical activity. With a single glance, the physician will be able to verify that the patient was indeed provided with adequate heart stimulation and that a proper cardiac electrical and mechanical response occurred during the MRI procedure. In addition to the use of visual indicators, or as an alternative thereto, an audio alarm could be used to generate an audio signal that represents the above danger condition. Still further, an MRI control signal could be generated as a result of the danger condition to disable or otherwise control the MRI equipment being used for the MRI procedure.

Accordingly, a photonic pacemaker-cardiac monitor has been disclosed that is particularly useful during MRI diagnostic procedures for stimulating an implanted heart while monitoring electrocardiographic “R” wave activity and/or mechanical activity. While various embodiments of the invention have been shown and described, it should be apparent that many variations and alternative embodiments could be implemented in accordance with the invention. For example, the indicator lights 5, 34 and 54 could be replaced with some other form of visual indicator, such as a meter, etc. In another modification, a photonic core body temperature monitor could be added to the apparatus 2 to provide additional sensing capability. To that end, a conventional thermister could be situated at the first distal housing 16. The thermister would be connected to a conventional bridge circuit that drives an electro-optical converter. The latter would send temperature-related optical information to the proximal end of the photonic catheter, where the optical signal would be converted by an opto-electrical converter into a corresponding electrical signal that drives a visual display.

It is understood, therefore, that the invention is not to be in any way limited except in accordance with the spirit of the appended claims and their equivalents.

I claim:

1. A photonic pacemaker-cardiac monitor apparatus, comprising:
a photonic catheter;
a photonic pacemaker adapted to pace a heart via said photonic catheter; and
a photonic electrocardiographic monitor adapted to sense cardiac electrical activity via said photonic catheter.

2. An apparatus in accordance with claim 1 further including a photonic oxygen monitor adapted to sense cardiac blood oxygen content via said photonic catheter.

3. An apparatus in accordance with claim 2 wherein said oxygen monitor comprises a Clark electrode.

4. An apparatus in accordance with claim 2 wherein said oxygen monitor comprises a pulse oximeter.

5. An apparatus in accordance with claim 2 further including a warning system for warning of a condition wherein one or more of the following occurs:
said patient fails to receive proper pacemaker stimulation;
said patient fails to exhibit proper cardiac electrical activity; or
said patient fails to exhibit proper cardiac mechanical activity.

6. An apparatus in accordance with claim 5 wherein said warning system includes a display mounted on a non-implantable control housing of said apparatus.

7. An apparatus in accordance with claim 5 wherein said display comprises a first visual indicator for providing an indication of said pacemaker generating a pulse, a second visual indicator for providing an indication of said electrocardiagraphic monitor sensing cardiac electrical activity, and a third visual indicator for providing an indication of said oxygen monitor sensing cardiac blood oxygen content.

8. An apparatus in accordance with claim 2 wherein said photonic catheter comprises optical conductors respectively associated with said pacemaker, said electrocardiagraphic monitor and said oxygen monitor.

9. An apparatus in accordance with claim 1 wherein said apparatus includes a hermetic housing at a distal end of said photonic catheter, said hermetic housing containing an opto-electrical converter associated with said pacemaker, an EKG amplifier and electro-optical converter associated with said electrocardiagraphic monitor, and an amplifier and electro-optical converter associated with said oxygen monitor.

10. An apparatus in accordance with claim 1 further including pacemaker feedback circuitry for adjusting said photonic pacemaker according to an output of said electrocardiographic monitor.

11. A photonic pacemaker-cardiac monitor apparatus for MRI diagnostic use, comprising:
a photonic catheter;
a photonic pacemaker adapted to pace a heart via said photonic catheter;
a photonic electrocardiographic monitor adapted to sense cardiac electrical activity via said photonic catheter;
a photonic oxygen monitor adapted to sense cardiac blood oxygen content via said photonic catheter;
said photonic oxygen monitor comprising one of a Clark electrode or a pulse oximeter;
a non-implantable control housing;
a warning system for warning of a condition wherein one or more of the following occurs:
said patient fails to receive proper pacemaker stimulation;
said patient fails to exhibit proper cardiac electrical activity; or
said patient fails to exhibit proper cardiac mechanical activity;
said warning system including a display located on said control housing for providing a visual indication of outputs from said pacemaker, said electrocardiographic monitor and said oxygen monitor;
said display comprising a first visual indicator for providing an indication of said pacemaker generating a pulse, a second visual indicator for providing an indication of said electrocardiagraphic monitor sensing cardiac electrical activity, and a third visual indicator for providing an indication of said oxygen monitor sensing cardiac blood oxygen content;
said photonic catheter comprising optical conductors respectively associated with said pacemaker, said electrocardiagraphic monitor and said oxygen monitor;
a hermetic housing at a distal end of said photonic catheter;
said hermetic housing containing an opto-electrical converter associated with said pacemaker, an EKG amplifier and electro-optical converter associated with said electrocardiagraphic monitor, and an amplifier and electro-optical converter associated with said oxygen monitor; and
pacemaker feedback circuitry for adjusting said photonic pacemaker according to an output of said electrocardiographic monitor.

12. A method for pacing and monitoring a patient undergoing an MRI procedure, comprising the steps of:
positioning a patient for an MRI procedure after said patient has been implanted with a photonic pacemaker-cardiac monitor apparatus, comprising:
a photonic catheter;
a photonic pacemaker adapted to pace a heart via said photonic catheter;
a photonic electrocardiographic monitor adapted to sense cardiac electrical activity via said photonic catheter;
a photonic oxygen monitor adapted to sense cardiac blood oxygen content via said photonic catheter;
a non-implantable control housing; and
a warning system for warning of a condition wherein one or more of the following occurs:
said patient fails to receive proper pacemaker stimulation;
said patient fails to exhibit proper cardiac electrical activity; or
said patient fails to exhibit proper cardiac mechanical activity;
commencing an MRI procedure on said patient;
monitoring said warning system while performing said MRI procedure; and
taking responsive action in the event that said warning system warns of said condition.
